(19) United States
(12) Patent Application Publication
(10) Pub. No.: US 2007/0135352 A1
(43) Pub. Date: Jun. 14, 2007

(54) Use of lh for generating libido and reaching orgasm in female subjects

(75) Inventor: Michael Jemec, Giubiasco (CH)

Correspondence Address: YOUNG & THOMPSON, 745 SOUTH 23RD STREET, 2ND FLOOR, ARLINGTON, VA, 22202, US

(73) Assignees: Michael Jemec, Giubiasco (CH); Studio Legale AVV. Aldo Ferrini E AVV. Manuele Bianchi, Lugano (CH)

(21) Appl. No.: 10/575,487

(22) PCT Filed: Sep. 16, 2004
(86) PCT No.: PCT/IB04/03045

§ 371 Date: Apr. 12, 2006

(30) Foreign Application Priority Data
Oct. 13, 2003 (GB) 01740/03

(51) Int. Cl. A61K 38/09

(52) U.S. Cl. 514/15

(57) ABSTRACT

The use is described of LH (Luteinizing Hormone) to cure a lack of or low libido and/or a difficulty in reaching orgasm in female subjects.

The present invention relates to the field of medicine, and more particularly endocrinology, and provides a novel solution to medical cases of gynaecological type concerning a lack of or low libido and/or an unnatural difficulty in reaching orgasm in female subjects.

It is well known that chronic pathological conditions in which the above symptoms are exhibited can seriously affect the emotional and family relationships of female subjects.

The pharmaceutical remedies used hitherto have in many cases proved to be inadequate solutions to the abovementioned problems: the inventors of the subject of the present application believed that there was a need for a novel solution, compatible with the natural physiology of women.

Being doctors and researchers, they knew that the female body secretes a particular hormone, LH (Luteinizing Hormone), a short time before ovulation. Given the chronological point at which this hormone appears in the menstrual cycle, its purpose is probably to make it more likely that intercourse will take place at the time of ovulation, by making the woman more inclined to have intercourse, as she is urged by the known physiological syndrome referred to as “libido” and by the instinctive desire for the sensations characteristic of an orgasm.

Based on these observations, the inventors believed that the administration to female subjects of synthesized LH could remedy a lack of or low libido and the difficulty in reaching orgasm.

Experiments carried out on many subjects confirmed the inventors' theory, and they therefore defined a novel use of the said LH that differs in terms of purpose from the other uses made of it hitherto, making it possible to provide efficient and physiologically natural solutions to the abovementioned problems.

The subject of the present invention is therefore the use of LH described in the attached claim 1.

The said LH may be administered parenterally or orally, if necessary by modifying its molecule beforehand so that it can also be assimilated through the mucous membranes in order to produce the characteristic effects of LH.

Oral administration preferably takes the form of tablets, but other vehicles, such as solutions or suspensions in the form of syrups or the like, may be used without any problems.

1-3. (canceled)

4. A method for treating a lack of or low libido and/or a difficulty in reaching orgasm in female subjects, comprising administering to said woman an effective amount of Luteinizing Hormone.

5. The method according to claim 4, in which the said hormone is administered parenterally.

6. The method according to claim 4, in which the said hormone is administered orally, by means of tablets.
